(19) United States
(12) Patent Application Publication
(10) Pub. No.: US 2024/0268645 A1
(43) Pub. Date: Aug. 15, 2024

(54) HYBRID FLUID SUPPLY SYSTEM FOR AN ENDOSCOPE

(71) Applicant: Boston Scientific Scimed, Inc., Maple Grove, MN (US)

(72) Inventors: Ryan V. Wales, Northborough, MA (US); John B. Golden, Norton, MA (US); Scott E. Brechbiel, Acton, MA (US); Austin Grant Johnson, Hudson, MA (US)

(73) Assignee: Boston Scientific Scimed, Inc., Maple Grove, MN (US)

(21) Appl. No.: 18/436,974

(22) Filed: Feb. 8, 2024

Related U.S. Application Data
(60) Provisional application No. 63/444,447, filed on Feb. 9, 2023.

(51) Int. Cl. A61B 1/015; A61B 1/00; A61B 1/12

(52) U.S. Cl. CPC A61B 1/015; A61B 1/00119; A61B 1/00128; A61B 1/126

(57) ABSTRACT

A fluid supply system for an endoscope includes a first container having an interior volume configured to contain a fluid, a second container having an interior volume configured to contain a fluid, and a coupling mechanism having a first portion and a second portion, and a lumen extending therebetween. The first portion of the coupling mechanism is configured to engage with the first container and the second portion is configured to engage with the second container. The first portion includes a first fluid port in fluid communication with the first container via the lumen, and the first container and the second container are in fluid communication via the lumen.

### CROSS-REFERENCE TO RELATED APPLICATIONS

This application claims the benefit of U.S. Provisional Patent Application Ser. No. 63/444,447 filed on Feb. 9, 2023, the disclosure of which is incorporated herein by reference.

### TECHNICAL FIELD

The present disclosure pertains to medical devices, and methods for manufacturing medical devices. More particularly, the present disclosure pertains to coupling mechanisms to supply fluid and/or gas to an endoscope.

### BACKGROUND

Endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. To enable these capabilities, compressed gasses from either the processor or alternative sources are used to increase the pressure within a fluid bottle, which either insufflates the working lumen, or washes the lens of the endoscope. Additionally, a peristaltic pump can be used to irrigate the working lumen of debris.

### BRIEF SUMMARY

The summary of the disclosure is given to aid understanding, and one of skill in the art will understand that each of the various aspects and features of the disclosure may advantageously be used separately in some instances, or in combination with other aspects and features of the disclosure in other instances. Accordingly, while the disclosure is presented in terms of aspects or embodiments, it should be appreciated that individual aspects can be claimed separately or in combination with aspects and features of that embodiment or any other embodiment.

In an example, a fluid supply system for an endoscope may include a first container having an interior volume configured to contain a fluid, a second container having an interior volume configured to contain a fluid, and a coupling mechanism having a first portion and a second portion, and a lumen extending therebetween, wherein the first portion of the coupling mechanism may be configured to engage with the first container and the second portion may be configured to engage with the second container. The first portion may include a first fluid port in fluid communication with the first container via the lumen, and the first container and the second container may be in fluid communication via the lumen.

Alternatively or additionally to any of the embodiments herein, the first container may be an intravenous (IV) bag, and an end of the first portion of the coupling mechanism may be a bag spike configured to be coupled to the IV bag.

Alternatively or additionally to any of the embodiments herein, the coupling mechanism may be a cap configured to cover an opening of the second container.

Alternatively or additionally to any of the embodiments herein, the first fluid port may be configured to engage with a first end of a first tubing, the first tubing having a second end configured to engage with the endoscope.

Alternatively or additionally to any of the embodiments herein, the second portion of the coupling mechanism may include a second fluid port configured to engage with a first end of a second tubing, the second tubing having a second end configured to engage with a fluid supply.

Alternatively or additionally to any of the embodiments herein, the cap may include a first spring and a second spring configured to actuate a sliding valve mechanism, the sliding valve mechanism configured to move from a first valve position to a second valve position.

Alternatively or additionally to any of the embodiments herein, the second container may be configured to receive a fluid from the first container through the lumen extending from the first portion to the second portion of the coupling mechanism based on a pressure at the second container.

Alternatively or additionally to any of the embodiments herein, the first container may be a first intravenous (IV) bag, and an end of the first portion of the coupling mechanism may include a first bag spike configured to be coupled to the first IV bag, and wherein the second container may be a second IV bag and an end of the second portion of the coupling mechanism may include a second bag spike configured to be coupled to the second IV bag.

Alternatively or additionally to any of the embodiments herein, a middle portion of the coupling mechanism may include the first fluid port, the middle portion positioned between the first portion and the second portion, wherein the first fluid port may be in fluid communication with the first container, the second container, and a third container via the lumen, and wherein the third container may be configured to contain a fluid.

Alternatively or additionally to any of the embodiments herein, the coupling mechanism may further include an adjustable valve positioned between the first portion and the second portion of the coupling mechanism, the adjustable valve may be configured to be adjusted to control a flow of fluid through the lumen extending between the first portion and the second portion.

In another example, a coupling mechanism for an endoscope may include a first portion configured to engage with a first container, a second portion configured to engage with a second container, a lumen extending from the first portion to the second portion, a first fluid port of the first portion, the first fluid port in fluid communication with the lumen, and a second fluid port of the second portion, the second fluid port is configured to be in fluid communication with a volume defined by the second container when the second portion engages the second container.

Alternatively or additionally to any of the embodiments herein, the second portion may include a valve configured to control a flow of fluid through the lumen extending from the first portion to the second portion.

Alternatively or additionally to any of the embodiments herein, the first container may be an intravenous (IV) bag, and the first end of the coupling mechanism may include a bag spike configured to be coupled to the IV bag.

Alternatively or additionally to any of the embodiments herein, the coupling mechanism may include a cap and the cap may include the first portion and the second portion.

Alternatively or additionally to any of the embodiments above, the cap may include a biasing system configured to actuate a sliding valve mechanism, the sliding valve mechanism may be configured to move from a first position to a second position in response to a pressure at the sliding valve mechanism, wherein the biasing system biases the sliding valve mechanism to the first position.

Alternatively or additionally to any of the embodiments herein, a fluid from the second fluid port may pressurize a space at the sliding valve mechanism, thereby causing the sliding valve mechanism to move from the first position to the second position.

In another example, a fluid supply system for an endoscope may include a first container having an interior volume configured to contain a fluid, a second container having an interior volume configured to contain a fluid, and a coupling mechanism having a first portion and a second portion, and a lumen extending therebetween, wherein the first end of the coupling mechanism may be configured to engage with the first container and the second end may be configured to engage with the second container. The first container and the second container may be in fluid communication via the lumen. The first portion of the coupling mechanism may further include a first fluid port configured to engage with a first end of a first tubing, the first tubing having a second end configured to engage with the endoscope, and the second portion of the coupling mechanism may further include a second fluid port configured to engage with a first end of a second tubing, the second tubing having a second end configured to engage with a fluid supply.

Alternatively or additionally to any of the embodiments herein, the second portion may include a valve configured to control a flow of fluid through the lumen.

Alternatively or additionally to any of the embodiments herein, the valve may include a sliding mechanism and the sliding mechanism may be configured to be actuated between a first position and a second position in response to a sensed pressure at the second portion of the coupling mechanism.

Alternatively or additionally to any of the embodiments herein, the valve may include a biasing system configured to actuate a sliding mechanism, the sliding mechanism may be configured to move from a first position to a second position in response to a pressure at the sliding mechanism, the biasing system biases the sliding mechanism to the first valve position, the first position may be configured to allow fluid to flow between the first container and the second container via the lumen, and the second position may be configured to block fluid flow between the first container and the second container via the lumen and allow fluid to flow from the second container through the second portion.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 depicts a schematic view of components of an illustrative endoscope;

FIG. 2 depicts a schematic view of components of an illustrative endoscope system;

FIG. 3A depicts a schematic view of an illustrative endoscope system, wherein the endoscope system is activated to deliver air to atmosphere;

FIG. 3B depicts a schematic view of an illustrative endoscope system, wherein the endoscope system is activated to deliver air to a patient through the patient end of the endoscope;

FIG. 3C depicts a schematic view of an illustrative endoscope system, wherein the endoscope system is activated to deliver lens wash fluid through the patient end of the endoscope;

FIG. 3D depicts a schematic view of an illustrative endoscope system, wherein the endoscope system is activated to deliver irrigation fluid through the patient end of the endoscope;

FIG. 4 depicts a schematic view of an illustrative hybrid endoscope system;

FIG. 5 depicts a schematic view of an illustrative fluid supply system for an endoscope system;

FIG. 6 depicts a schematic view of an illustrative coupling mechanism for a fluid supply;

FIG. 7 depicts a schematic bottom view of the illustrative coupling mechanism of FIG. 6;

FIG. 8 depicts a schematic cross-sectional view of the illustrative coupling mechanism of FIG. 6;

FIG. 9 depicts a schematic bottom view of an illustrative coupling mechanism including a cap insert;

FIG. 10A depicts a schematic cross-sectional view of the illustrative coupling mechanism of FIG. 9 including the cap insert, wherein a sliding mechanism is in a first position;

FIG. 10B depicts a schematic cross-sectional view of the illustrative coupling mechanism of FIG. 10A including the cap insert, wherein the sliding mechanism is in a second position;

FIG. 11A depicts a schematic cross-sectional view of an illustrative coupling mechanism including a sliding mechanism, wherein the sliding mechanism is in a first position;

FIG. 11B depicts a schematic cross-sectional view of the illustrative coupling mechanism including the sliding mechanism of FIG. 11A, wherein the sliding mechanism is in a second position;

FIG. 12 depicts a schematic view of an illustrative coupling mechanism;

FIG. 13A depicts a schematic cross-sectional view of the illustrative coupling mechanism of FIG. 12, wherein an adjustable valve is in a first position;

FIG. 13B depicts a schematic cross-sectional view of the illustrative coupling mechanism of FIG. 12, wherein an adjustable valve is in a second position;

FIG. 13C depicts a schematic cross-sectional view of the illustrative coupling mechanism of FIG. 12, wherein an adjustable valve is in a third position; and

FIG. 14 depicts a schematic view of an illustrative fluid supply system for an endoscope system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

Endoscope devices are used for performing diagnostic and/or therapeutic treatments. During endoscopic procedures, physicians may use a combination of air, irrigation and lens wash as a means of flushing debris, cleaning optics, and insufflating the working lumen. To enable these capabilities compressed gasses from either the processor or alternative source are used to increase the pressure within a fluid bottle which either insufflates the working lumen or wash the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day which may introduce multiple opportunities for contamination to the tube set by either contacting non-sterile surfaces or dropping the tubing on the floor. Disclosed herein are methods and systems to reduce or eliminate the need to disconnect the tube set and use a second bottle.

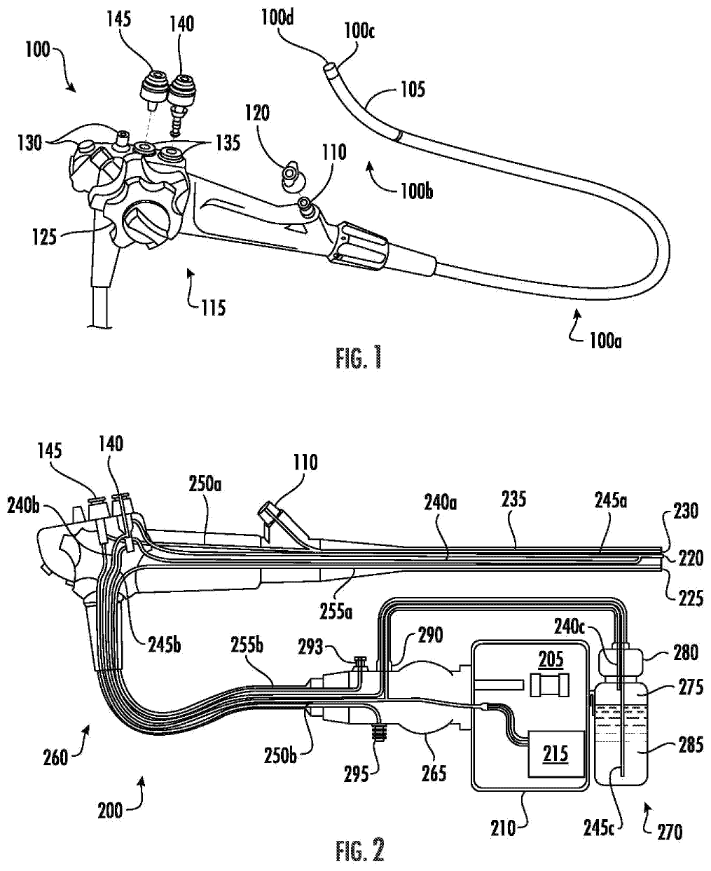

With reference to FIGS. 1-2, an illustrative endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO2) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an illustrative operation of an endoscopic system 200, including an endoscope such as endoscope 100 above, is explained. Air from the air pump 215 in the video processing unit 210 is flowed through the connection portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

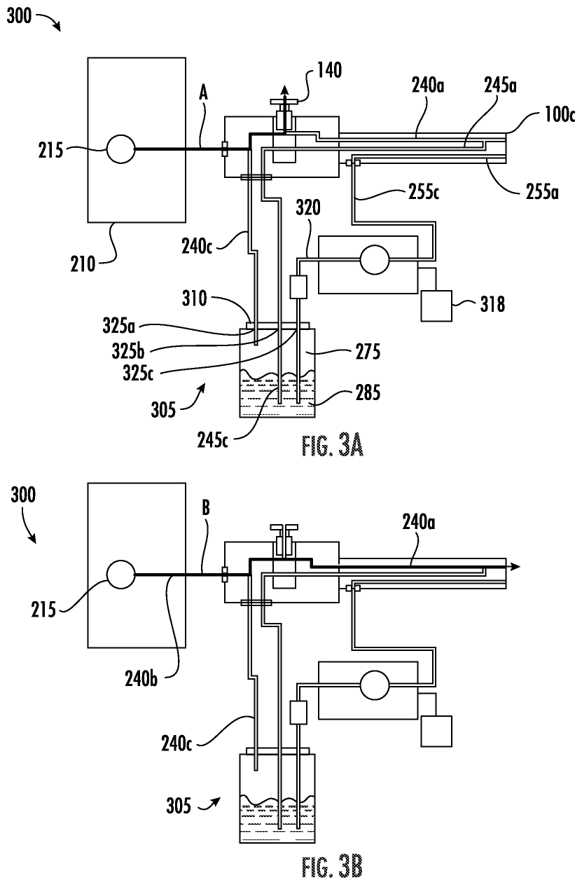

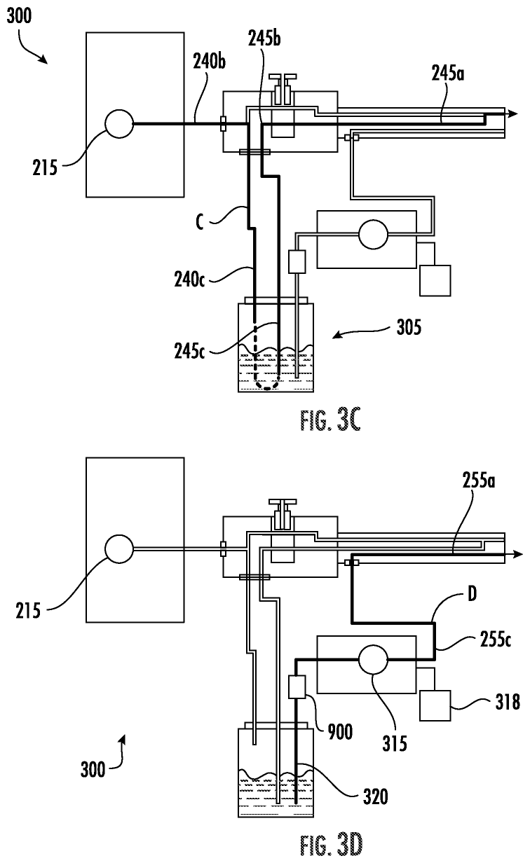

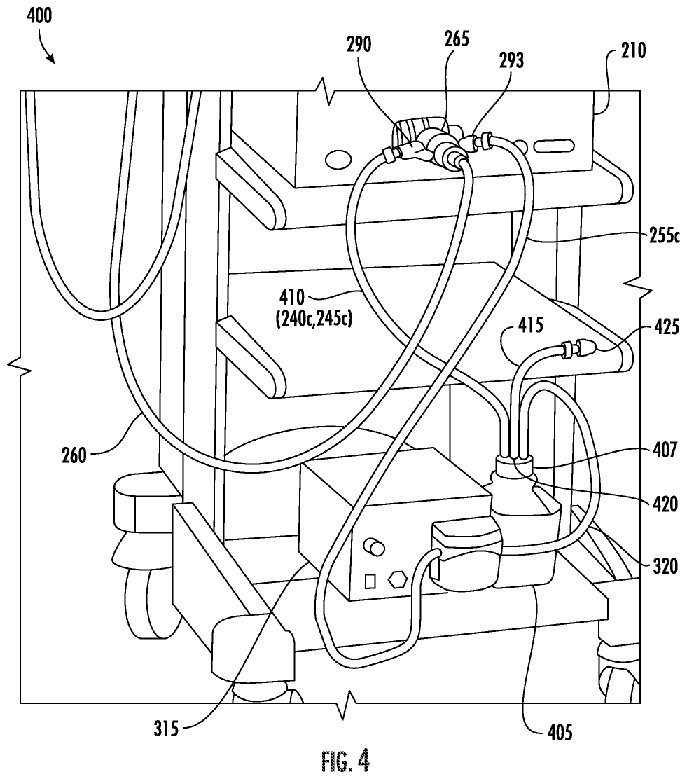

FIGS. 3A-3D are schematic drawings illustrating the operation of an embodiment of a hybrid system 300 where the supply tubing for irrigation and lens wash are connected to and drawn from a single water reservoir. It is contemplated that fluids other than water may be used, such as, but not limited to saline. The hybrid system 300 includes the single water reservoir 305, a cap 310 for the reservoir, gas supply tubing 240c, lens wash supply tubing 245c, irrigation pump 315 with foot switch 318, upstream irrigation tubing 320 and downstream irrigation supply tubing 255c. The cap 310 may be configured to attach in a seal-tight manner to the water reservoir 305 by a typically threaded arrangement. The cap 310 may include a gasket to seal the cap 310 to the reservoir 305. The gasket can be an O-ring, flange, collar, and/or the like and can be formed of any suitable material. A number of through-openings (325a, 325b, 325c) in the cap 310 are provided to receive, respectively, the gas supply tubing 240c, lens wash supply tubing 245c, and upstream irrigation supply tubing 320. In FIGS. 3A-3D, the system depicted includes separate tubing for gas supply, lens wash, and irrigation.

In other embodiments, the gas supply tubing 240c and lens wash tubing 245c may be combined in a coaxial arrangement. For example, the gas supply tubing may define a lumen that is sufficiently large in diameter to encompass a smaller diameter lens wash tubing, coaxially received within the gas supply tubing, as well as provide air to the water source in an annular space surrounding the lens wash tubing to pressurize the water reservoir (see, e.g., gas and lens wash supply tubing 240c, 245c). Some illustrative coaxial arrangements are described in commonly assigned U.S. patent application Ser. No. 17/558,239, titled INTEGRATED CONTAINER AND TUBE SET FOR FLUID DELIVERY WITH AN ENDOSCOPE and U.S. patent application Ser. No. 17/558,256, titled TUBING ASSEMBLIES AND METHODS FOR FLUID DELIVERY, the disclosures of which are hereby incorporated by reference in their entirety for any and all purposes. The lens wash supply tubing may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion (e.g., connector portion 265 of FIG. 2).

In various embodiments, different configurations of valving (not shown) may be incorporated into various embodiments disclosed hereby, including the tubing of the system 200, 300. For example, an in-flow check valve can be disposed in the path of the gas supply tubing 240c to help prevent backflow into the air pump 215. In this manner, pressure building within the water reservoir 305 creates a pressure difference between the water source and the gas supply tubing 240c helping to maintain a positive pressure in the water source even when large amounts of water may be removed from the water source during the irrigation function. This arrangement compensates for any time lag in air being delivered from the air pump 215 to the water reservoir 305, which might otherwise cause a negative pressure vacuum in the water reservoir. Similarly, an out-flow check valve, such as the one-way valve with inlet/outlets and valve insert, may be incorporated in the lens wash supply tubing 240c, upstream irrigation supply tubing 320, and/or downstream irrigation supply tubing 255c to help prevent backflow of water from either or both of the lens wash and irrigation tubing in the event of a negative pressure situation, as described.

More generally, in many embodiments, a check valve may refer to any type of configuration for fluid to flow only in one direction in a passive manner. For example, a check valve may include, or refer to, one or more of a ball check valve, a diaphragm check valve, a swing check valve, a tilting disc check valve, a flapper valve, a stop-check valve, a lift-check valve, an in-line check valve, a duckbill valve, a pneumatic non-return valve, a reed valve, a flow check. Accordingly, a check valve as used herein is meant to be separate and distinct from an active valve that is operated in a binary manner as an on/off valve or switch to allowed flow to be turned on or allow flow to be turned off (e.g., a stop cock valve, solenoid valve, peristaltic pump).

During operation of the system of FIGS. 3A-3D, a flow of water for irrigation may be achieved by operating the irrigation pump 315. A flow of water for lens wash may be achieved by depressing the gas/water valve 140 on the operating handle 115 of the endoscope 100. These functions may be performed independent of one another or simultaneously. When operating lens wash and irrigation at the same time, as fluid is removed from the water reservoir 305, the pressure in the system may be controlled to maintain the lens wash supply tubing 240c at substantially the pressure necessary to accomplish a lower flow rate lens wash, while compensating for reduced pressure in the water reservoir 305 due to supplying a high flow rate irrigation. When pressure is reduced in the water reservoir by use of the lens wash function, the irrigation function, or both functions simultaneously, the reduced pressure may be compensated for by the air pump 215 via the gas supply tubing 240c.

The schematic set-up in FIGS. 3A-3D has been highlighted to show the different flow paths possible with the hybrid system 300 having supply tubing for irrigation 320 and lens wash 240c connected to and drawn from the single water reservoir 305. As shown in FIG. 3A, the endoscope 100 is in a neutral state with the gas/water valve 140 in an open position. The neutral state delivers neither gas, nor lens wash, to the distal tip of the endoscope. Rather gas (pressure) is delivered along path A from the pressurizing air pump 215 and vented through the gas feed line 240b in the umbilical 260 via the connector portion 265 and through the gas/water valve to atmosphere. Since the system is open at the vent hole in the gas/water valve 140, there is no build up to pressurize the water reservoir 305 and consequently no water is pushed through the lens wash supply tubing 240c.

As shown in FIG. 3B, the endoscope 100 is in a gas delivery state with the gas/water valve 140 in a first position. When gas is called for at the distal tip 100c, for example, to clean the end face 100d of the distal tip or insufflate the patient body in the treatment area, the user closes off the vent hole in the gas/water valve 140 with a thumb, finger, or the like (first position). In this state, gas (pressure) is delivered along path B from the air pump 215 and flowed through the gas feed line 240b in the umbilical 260 via the connector portion 265. The gas continues through the gas/water valve 140 to the gas supply line 240a in the endoscope shaft 100a and out the gas/lens wash nozzle 220 at the distal tip 100c. There is no build up to pressurize the water reservoir since the system is open at the gas/lens water nozzle 220, and consequently no water is pushed through the lens wash supply tubing 240c.

As shown in FIG. 3C, the endoscope 100 is in a lens wash delivery state with the gas/water valve 140 in a second position. When lens wash is called for at the distal tip 100c, for example, to clean the end face 100d of the distal tip 100c, the user, keeping the vent hole in the air/water valve closed off, depresses the valve 140 to its furthest point in the valve well 135. The second position blocks off the gas supply to both atmosphere and the gas supply line 240a in the endoscope, and opens up the gas/water valve 140 to allow lens wash water to pass through to the lens wash supply line 245a in the endoscope shaft 100a and out the gas/lens wash nozzle 220 at the distal tip 100c. In this state, gas (pressure) is delivered along path C from the air pump 215, through the branched line in the connector portion 265 and out of the gas supply tubing 240c to the water reservoir 305. The gas (pressure) pressurizes the surface of the remaining water 285 in the reservoir 305 and pushes water up the lens wash supply tube 245c to the connector portion 265. The pressurized lens wash water is pushed further through the lens wash feed line 245b in the umbilical 260 and through the gas/water valve 140. Since the system 300 is closed, gas pressure is allowed to build and maintain a calibrated pressure level in the water reservoir 305, rather than venting to atmosphere or being delivered to the patient. This pressure, along with the endoscope feed and supply lines and external tubing, translates to a certain range of flow rate of the lens wash.

As shown in FIG. 3D, the endoscope 100 is in an irrigation delivery state. This may be performed at the same or a different time from the delivery of gas and/or lens wash. When irrigation is called for at the distal tip 100c, for example, if visibility in the treatment area is poor or blocked by debris, or the like, the user activates the irrigation pump 315 (e.g., by depressing foot switch 318) to delivery water along path D. With the pump 315 activated, water is sucked out of the water reservoir 305 through the upstream irrigation supply tubing 320 and pumped along the downstream irrigation supply tubing 255c to the connector portion 265. The irrigation pump head pressure pushes the irrigation water further through the irrigation feed line 255b in the umbilical 260, through the irrigation supply line 255a in the endoscope shaft 100a, and out the irrigation opening 225 at the distal tip 100c. The irrigation pump pressure may be calibrated, along with the endoscope irrigation feed and supply lines and external tubing, to deliver a certain range of flow rate of the irrigation fluid.

FIG. 4 is a schematic drawing illustrating a further embodiment of a hybrid system 400 including a video processing unit 210, connector portion 265, peristaltic irrigation pump 315, water reservoir 405 and top 407, coaxial gas and lens wash supply tubing 410, upstream and downstream irrigation supply tubing 320, 255c, and alternative gas supply tubing 415 (e.g., CO₂). A length of the alternative gas supply tubing 415 passes from one end positioned in the gas gap 275 between the top 407 of the water reservoir 405 and the remaining water 285 in the reservoir through an additional opening 420 in the top of the reservoir to a detachable connection 425 for a source of the alternative gas supply (e.g., CO₂ hospital house gas source). When the alternative gas supply is desired, such as CO₂ gas, the air pump 215 on the video processing unit 210 may be turned off and CO₂ gas, rather than air, is thereby flowed to the water reservoir 405 pressurizing the water surface. In the neutral state, CO₂ gas flows backward up the gas supply tubing 240c to the connector portion 265, up the gas feed line 240b, and is vented through the gas/water valve 140 to atmosphere. In the first position, the user closes off the vent hole in the gas/water valve 140, and the CO₂ gas is flowed through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the reservoir 405 is maintained by delivery gas through alternative gas (e.g., CO₂) supply tubing 415. The irrigation function may be accomplished in a similar manner as the operation described above with respect to FIG. 3D.

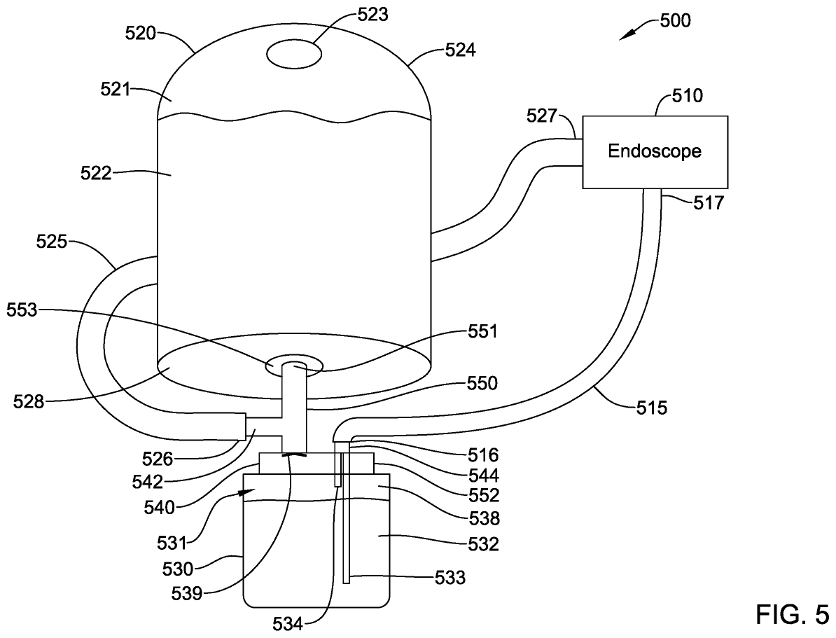

As described above, it may be desirable to reduce opportunities for contamination to the tube set 240c, 245c, 320, 410, 415 during replacement of the water reservoir by providing a larger volume water reservoir and/or a refillable water reservoir. FIG. 5 depicts a schematic view of an illustrative fluid supply system 500 for an endoscope 510. The fluid supply system 500 may include a first container 520 having an interior volume 521 configured to contain a fluid 532 (e.g., water). In some cases, the first container 520 may be formed from a lightweight, flexible material, such as, but not limited to low density polyethylene (LDPE), thermoplastic polyurethane (TPU), silicone, polyethylene terephthalate (PET), aluminum, nylon, polyethylene (PE), or combinations thereof, etc. In one example, the first container 520 formed from a lightweight, flexible material may be an IV (intravenous) bag. Additionally or alternatively, the first container 520 may be formed from a rigid or semi-rigid material. In some cases, the first container 520 may be entirely translucent, entirely opaque, or combinations thereof.

The first container 520 may include a carrying handle or hanging hook positioned adjacent to a top portion 524 thereof. The handle 523 may define an opening or through hole for receiving a hand or hook therethrough to carry or otherwise support the first container 520. In some embodiments, the handle 523 may be configured to couple to a hook or other mechanism on the endoscope tower. In some cases, the first container 520 may be coupled to the tower via hanging loop or hook and loop closures. This may elevate the first container 520 to reduce a footprint on floor of the procedure room or other flat surface and improve ergonomics since the user may no longer need to bend down to the floor to interface with the first container 520.

The fluid supply system 500 may include a second container 530 having an interior volume 531 which may be configured to contain a fluid 532 (e.g., water). In some cases, the second container 530 may be formed from a lightweight, flexible material, such as, but not limited to low density polyethylene (LDPE), thermoplastic polyurethane (TPU), silicone, polyethylene terephthalate (PET), aluminum, nylon, polyethylene (PE), or combinations thereof, etc. Additionally or alternatively, the second container 530 may be formed from a rigid or semi-rigid material. In some cases, as shown in FIG. 5, the second container 530 may be a water bottle. Additionally or alternatively, it is contemplated that the second container 530 may be a second IV bag and/or other suitable container(s). In some cases, the second container 530 may be entirely translucent, entirely opaque, or combinations thereof.

The first container 520 may be fluidly coupled to the second container 530 via a coupling mechanism 550. In some cases, the coupling mechanism 550 may include a first portion 551 and a second portion 552 with a lumen (not explicitly shown) extending therebetween.

The first portion 551 of the coupling mechanism 550 may be configured to engage with a spike port 553 positioned at a bottom portion 528 of the first container 520. For example, the first portion 551 may include a bag spike which may be configured to be inserted into the spike port 553 of the first container 520.

The first portion 551 of the coupling mechanism 550 may further include a first fluid port 542 which may be in fluid communication with the first container 520 via the lumen. The first fluid port 542 may be in fluid communication with the endoscope 510 via an irrigation supply tubing 525 (e.g., a first tubing) and/or other suitable tubing.

The irrigation supply tubing 525 may include a first end 526 and a second end 257. The first end 526 of the irrigation supply tubing 525 may be configured to engage with the first fluid port 542 via a hose barb mechanism. In some cases, the first end 526 of the irrigation supply tubing 525 may be configured to engage with the first fluid port 542 via interference fit, a luer lock system, a luer slip system, or any other suitable type of engagement. The second end 527 of the irrigation supply tubing 525 may be configured to engage with the endoscope 510 via a hose barb mechanism. In some cases, the second end 527 of the irrigation supply tubing 525 may be configured to engage with the endoscope 510 via interference fit, a luer lock system, a luer slip system, or any other suitable type of engagement.

The second portion 552 of the coupling mechanism 550 may include a cap 540. The cap 540 may be configured to cover an opening of the second container 530. The cap 540 at and/or of the second portion 552 may be fitted for the second container (e.g., a bottle) 530, and may be configured to couple to the second container 530 via a thread fastening mechanism and/or other suitable connecting mechanism.

The second portion 552 of the coupling mechanism 550 may further include a second fluid port 544. The second fluid port 544 may be in fluid communication with the interior volume 531 of the second container 530. A first end 516 of a second tubing 515 may be coupled to the second fluid port 544 and a second end 517 of the second tubing 515 may be coupled to the endoscope 510. The second tubing 515 may be considered to be a shared tubing, which may include a dual lumen, but this is not required.

The second container 530 may be in fluid communication with the endoscope via a shared length of gas supply tubing 534 and a lens wash supply/water supply tubing 533 and/or other suitable tubing. The shared length of gas supply tubing 534 may extend from the second fluid port 544 in the second portion 552 of the coupling mechanism 550. The shared length of gas supply tubing 534 may terminate within a container gap 538, at or below the opening of the second fluid port 544, but not extending into the remaining fluid 532 in the second container 530. However, in some cases, the gas supply tubing 534 may extend into the fluid 532. For example, the opening may be at a bottom or side of the second container 530 such that the shared gas supply tubing 534 terminates within the fluid 532 with gas bubbling up through the fluid 532 to pressurize the second container 530.

A lumen may extend through the gas supply tubing 534 for receiving a flow of air and/or gas therethrough. The lumen of the gas supply tubing 534 may be in operative fluid communication with the interior volume 531 of the second container 530.

The lens wash supply/water supply tubing 533 may extend from a second end external to the second container 530 through the second fluid port 544, terminating in a first end within the remaining fluid 532 at or substantially at the bottom of the second container 530. In some embodiments, the lens wash supply/water supply tubing 533 may terminate at the second fluid port 544. For example, when the opening is at or adjacent to the bottom portion of the second container 530 a dip tube of or separate from the water supply tubing may not be required.

A lumen may extend through the lens wash supply/water supply tubing 533 for receiving a flow of fluid therethrough. The lumen of the lens wash supply/water supply tubing 533 may be in selective operative fluid communication with the bottom portion of the second container 530.

In the illustrated configuration of FIG. 5, the gas supply tubing 534 and the lens wash supply/water supply tubing 533 may enter the second container 530 through a single or common tubing, (e.g., second tubing 515). For example, the gas supply tubing 534 and the lens wash supply/water supply tubing 533 may be coaxially arranged as the second tubing 515. However, this is not required. In some cases, the gas supply tubing 534 and the lens wash supply/water supply tubing 533 may extend in a side by side arrangement, may be separately connected to the second container 530 in different locations, and/or may have one or more other suitable configurations.

The first end 516 of the second tubing 515 may be configured to engage with the second fluid port 544 in any suitable manner. In some cases, the first end 516 of the second tubing 515 may be configured to engage with the second fluid port 544 via interference fit, a barb fit, a luer lock system, a luer slip system, and/or any other suitable type of engagement. In one example, the first end 516 of the second tubing 515 may be coupled to the second fluid port 544 via a hose barb mechanism.

The second end 517 of the second tubing 515 may be configured to engage with the endoscope 510 in any suitable manner. In some cases, the second end 517 of the second tubing 515 may be configured to engage with the endoscope 510 via interference fit, a barb fit, a luer lock system, a luer slip system, or any other suitable type of engagement. In one example, the second end 517 of the second tubing 515 may be coupled to the endoscope 510 via a hose barb mechanism.

It is contemplated that the second container 530 may be filled and refilled as needed by coupling the second container 530 to the first container 520. The refilling of the second container 530 may be performed during a procedure or between procedures, as needed. The fluid (e.g., water) may be sterile or non-sterile, as desired. In some cases, it may be desirable to refill the second container 530 without having to tip or pour a fluid source. Further, it may be desirable to refill the second container 530 to reduce the risk of contamination that may occur when changing out containers and/or tubing.

The first container 520 may be in fluid communication with the second container 530 via the lumen of the coupling mechanism 550, as discussed. In some cases, the second container 530 may be configured to receive a fluid (e.g., fluid 522) from the first container 520 via the lumen extending from the first portion 551 to the second portion 552 of the coupling mechanism 550 based on a pressure at the second container 530 and/or a pressure differential between the first container 520 and the second container 530. For example, when the interior volume 531 of the second container 530 is depressurized, such as, in-between procedures, a valve 539 within the second portion 552 of the coupling mechanism 550 may give way to the fluid 522 flowing through the lumen of the coupling mechanism 550 from the first container 520 and into the second container 530.

In some cases, the valve 539 may be an adjustable valve such as an umbrella valve, but this is not required. Additionally or alternatively, the valve 539 may be a duckbill valve, a one-way valve, a dome valve, or any other suitable valve.

In some cases, when the fluid 532 in the second container 530 is pressurized, the pressure within the interior volume 531 of the second container 530 increases and the valve 539 closes, thereby stopping a flow of fluid through the lumen of the coupling mechanism 550 from the first container 520 and into the second container 530. In other cases, when a user calls for irrigation at the endoscope 510, a suction through the irrigation supply tubing 525 pulls the fluid from the first container 520 through the first fluid port 542, and the suction closes the valve 539, thereby stopping a flow of fluid through the lumen of the coupling mechanism 550 from the first container 520 and into the second container 530, and redirecting the fluid through the irrigation supply tubing 525 to the endoscope 510.

In some cases, the gas supply tubing 534 may facilitate a gas (e.g., air, CO₂, etc.) flow from a gas supply (not explicitly shown) through the gas supply tubing 534 and into the interior volume 531 of the second container 530. This may allow the gas to pressurize the fluid 532 within the second container 530, thereby closing the valve 539, and forcing the fluid 532 out through the water supply tubing 533 and through a lens wash supply tube and/or an irrigation supply tube.

When the water supply tubing 533 is connected to or part of a lens wash supply tube, the volume of the flow through the lens wash supply tube may be governed by a gas pressure in the second container 530. When gas pressure begins to drop in the second container 530, (e.g., as water is pushed out of the second container 530 through the lens was supply/water supply tubing 533), the gas supply tubing 534 may supply gas to the second container 530 that replaces a volume of water removed from the second container 530 to maintain a substantially constant pressure in the second container 530, which in turn provides for a substantially constant lens wash flow rate.

In some configurations, a filter (not shown) may be placed in the path of the gas supply tubing 534 to filter-out undesired contaminants or particulates from passing into the second container 530. In some embodiments, outflow check valves or other one-way valve configurations (not shown) may be placed in the path of the lens wash supply/water supply tubing 533 to help prevent water from backflowing into the second container 530 after the water has passed the valve.

In some cases, a relatively higher flow rate of irrigation water may be required compared to lens wash since a primary use of the irrigation system is to clear the treatment area in the patient of debris that obstructs the user's field of view. In some cases, irrigation may be achieved with the use of a pump (e.g., peristaltic pump) in communication with the irrigation supply tubing 525.

In some cases, in order to equalize the pressure in the second container 530 as water is pumped out of the lens wash supply/water supply tubing 533 and/or the irrigation supply tubing 525, an air vent (not shown) may be included in the cap 540 of the second portion 552 of the coupling mechanism 550 and/or at one or more other suitable location of the first container 520, the second container, 530, and/or the coupling mechanism 550. The vent may allow atmospheric air into the water source preventing negative pressure build-up in the water source, which could create a vacuum that suctions undesired matter from the patient back through the endoscope toward the water source. In some embodiments, outflow check valves or other one-way valve configurations (not shown) may be placed in the path of the irrigation supply tube 525 and/or the lens wash supply/water supply tubing 533 to help prevent back-flow into the second container 530.

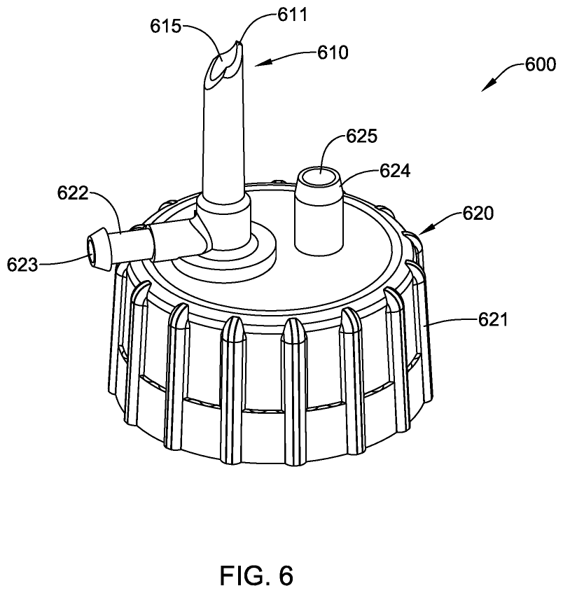

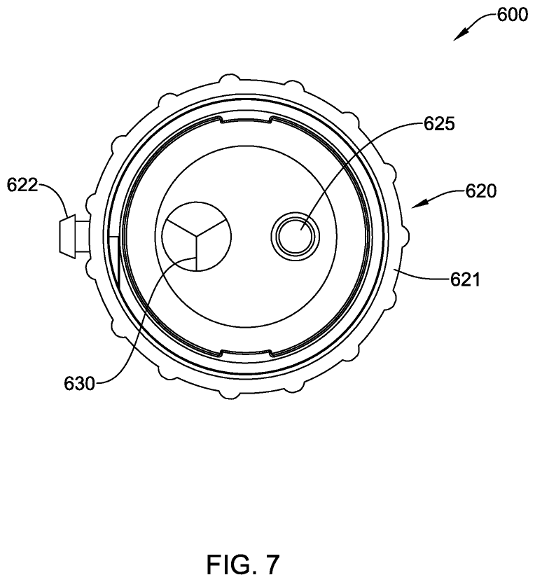

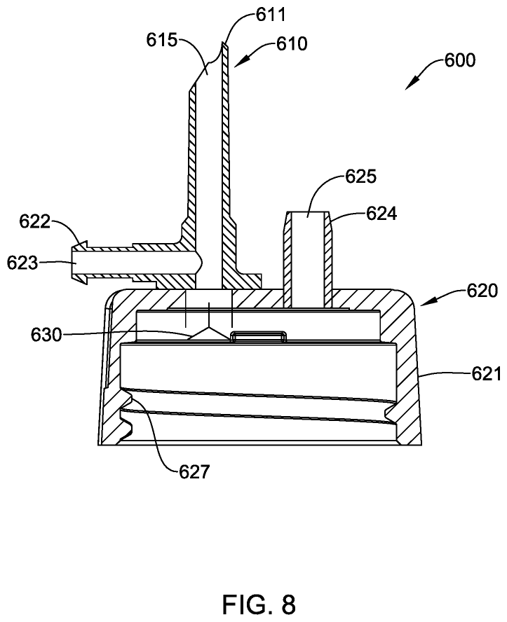

FIG. 6 depicts a schematic perspective view of an illustrative coupling mechanism 600 for a fluid supply, FIG. 7 depicts a schematic bottom view of the illustrative coupling mechanism 600, and FIG. 8 depicts a schematic cross-sectional view of the illustrative coupling mechanism 600. The coupling mechanism 600 may be considered to be an example of the coupling mechanism 550.

As shown in FIGS. 6 to 8, the coupling mechanism 600 may include a first portion 610, a second portion 620, and a lumen 615 extending through the first portion 610 and the second portion 620. An end 611 of the first portion 610 may be and/or may include a bag spike configured to engage with a spike port of an IV bag (e.g., the first container 520 and/or other suitable container). The second portion 620 of the coupling mechanism 600 may be a cap 621 configured to cover an opening of a container (e.g., the second container 530 and/or other suitable container).

The cap 621 may be configured to be coupled to a container via a snap fit, interference fit, or may include a heat seal to seal the container in a fluid and pressure tight manner. In one example, the cap 621 may be configured to be coupled to a container, such as a bottle, via a thread fastening mechanism 627, as shown in FIG. 8.

The coupling mechanism 600 may be made from any suitable materials including, but not limited to, polymers, metals, combinations of material types, and/or other suitable materials. In one example, the coupling mechanism 600 may be entirely or at least partially formed from one or more polycarbonate materials. Where polymers are selected as materials for the coupling mechanism 600, the materials may have a durometer in a range of about 75A to 90A Shore hardness, among other possible values.

The first portion 610 of the coupling mechanism 600 may include a first fluid port 622. The first fluid port 622 may include a lumen 623 that may be in fluid communication with the lumen 615. The first fluid port 622 may be configured to engage with an irrigation supply tubing (e.g., irrigation supply tubing 525 and/or other suitable irrigation supply tubing). The first fluid port 622 may be configured to engage with an irrigation supply tubing via an interference fit, a one way locking feature, adhesive bonding, crimping, or the like. In some cases, the first fluid port 622 may include a barb feature, as shown in FIG. 6. The second portion 620 of the coupling mechanism 600 may include a second fluid port 624. The second fluid port 624 may be in fluid communication with a second container via a lumen 625. The second fluid port 624 may be configured to engage with a second tubing (e.g., second tubing 515 and/or other suitable tubing) which may house a gas supply tubing (e.g., gas supply tubing 534) and/or a water supply tubing (e.g., water supply tubing 533), as discussed with reference to FIG. 5.

The coupling mechanism 600 may be configured to fluidly couple a first container (e.g., an IV bag) and a second container (e.g., a water bottle) via the lumen 615. In some cases, the second container coupled to the second portion 620 of the coupling mechanism 600 may be configured to receive a fluid (e.g., water) from the first container coupled to the first portion 610 via the lumen 615 extending from the first portion 610 to the second portion 620 of the coupling mechanism 600 based on a pressure at the second container (e.g., second container 530). For example, when an interior volume of the second container is depressurized, such as, in-between procedures, a valve 630 within the second portion 620 of the coupling mechanism 600 may be configured to give way to the fluid flowing through the lumen 615 of the coupling mechanism 600 from the first container and into the second container. In some cases, as the second container is pressurized, the pressure within the interior volume may cause the valve 630 to close, thereby stopping a flow of fluid through the lumen 615 of the coupling mechanism 600 from the first container and into the second container. In other cases, when a user calls for irrigation at an endoscope, a suction through the first tubing pulls the fluid from the first container through the first fluid port 622, and the suction closes the valve 630, thereby stopping a flow of fluid through the lumen 615 of the coupling mechanism 600 from the first container and into the second container, and redirecting the fluid through the first tubing to the endoscope.

The valve 630 may be any suitable type of valve. For example, the valve 630 may be an adjustable valve, an F valve, a duckbill valve, a one-way valve, and/or other any other suitable type of valve. In one example, the valve 630 may be an automatically adjustable umbrella valve configured to adjust based on one or more adjacent pressures.

In some cases, the gas supply tubing coupled to the second fluid port 624 may allow a gas (e.g., air, CO₂) to flow from a gas supply (not explicitly shown) through the gas supply tubing and into the interior volume of the second container. This allows the gas to pressurize the fluid within the second container, thereby closing the valve 630, and forcing the fluid out through the second fluid port 624 and the lens wash supply/water supply tubing. When gas pressure begins to drop in the second container, as water is pushed out of the second container through the lens wash supply/water supply tubing, the gas (e.g., via the gas supply tubing) may replace a volume of water outputted from the second container to maintain a substantially constant pressure, which in turn provides for a substantially constant lens wash flow rate.

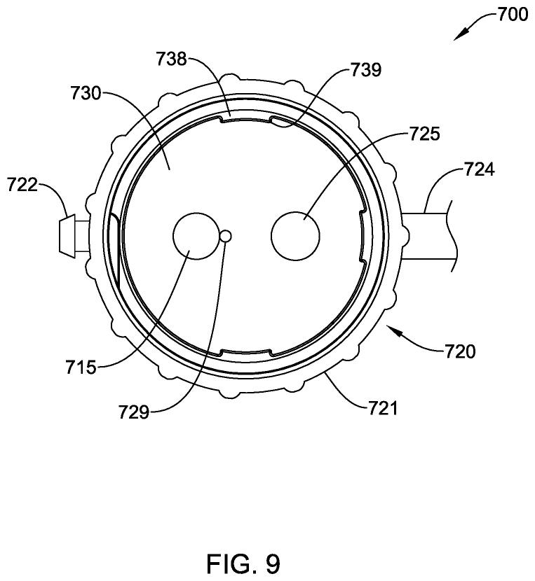

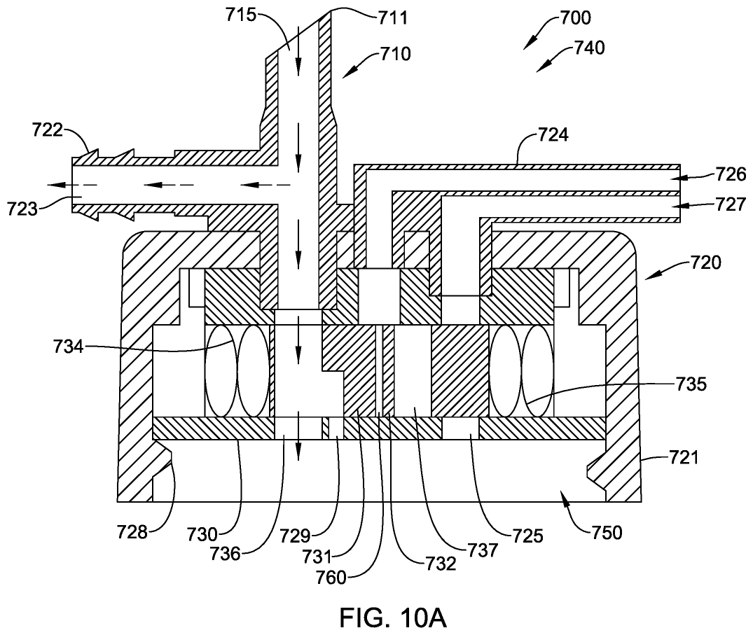

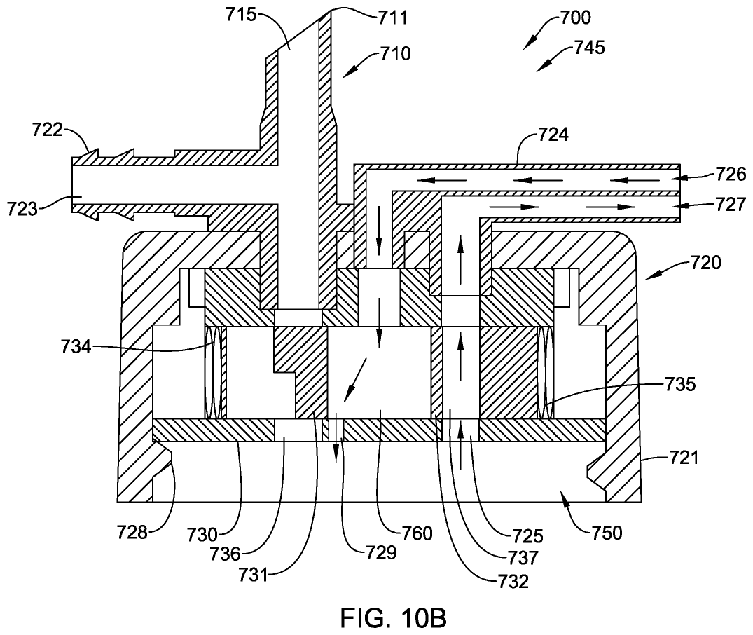

FIG. 9 depicts a bottom view of an illustrative coupling mechanism 700 including a cap insert 730, FIG. 10A depicts a cross-sectional view of the illustrative coupling mechanism 700, and FIG. 10B depicts a cross-sectional view of the illustrative coupling mechanism 700. The coupling mechanism 700 may include a first portion 710, a second portion 720, and a lumen 715 extending through the first portion 710 and the second portion 720. An end 711 of the first portion 710 may include and/or may be a bag spike configured to engage with a spike port of an IV bag (e.g., first container 520 and/or other suitable container). The second portion 720 of the coupling mechanism 700 may be a cap 721 configured to cover an opening of a container (e.g., second container 530 and/or other suitable container). Other suitable configurations of the coupling mechanism 700 are contemplated.

The cap 721 may be configured to couple to a container in any suitable manner. In some cases, the cap 721 may be configured to be coupled to a container via a snap fit, a threaded connection, an interference fit, a heat seal to seal the container in a fluid and pressure tight manner, and/or via other suitable coupling techniques. In one example, the cap 721 may be configured to couple to a container, such as a bottle, via a thread fastening mechanism 728.

The coupling mechanism 700 may be formed from any suitable materials including, but not limited to, polymers, metals, combinations of materials, and/or other suitable materials. In one example, the coupling mechanism 700 may be entirely or at least partially formed from polycarbonate materials. Where polymers are selected as coupling mechanism 700 materials, the materials may have durometer in a range of about 75A to 90A Shore hardness, among other possible values.

The first portion 710 of the coupling mechanism 700 may include a first fluid port 722 and/or other suitable fluid ports. The first fluid port 722 may include a lumen 723 that may be in fluid communication with the lumen 715. The first fluid port 722 may be configured to engage with an irrigation supply tubing (e.g., irrigation supply tubing 525) and/or other suitable tubing. In some cases, when irrigation is called for, fluid from the first container may flow through the lumen 715 and may be directed to the lumen 723, as indicated by the dashed arrows.

The second portion 720 of the coupling mechanism 700 may include a second fluid port 724 and/or other suitable ports. The second fluid port 724 may be in fluid communication with a second container via an opening 725. The second fluid port 724 may be configured to engage with a second tubing (e.g., second tubing 515) and/or other suitable tubing which may house a gas supply tubing 726 and a water supply tubing 727, as discussed with reference to FIG. 5.

As shown in FIGS. 9 to 10B, the coupling mechanism 700 may include the cap insert 730. The cap insert 730 may be positioned within the cap 721 via a bayonet fit in which one or more notches 739 of the cap insert 730 align with one or more tabs 738 of the second portion 720 of the coupling mechanism 700 for insertion and the cap insert 730 may be rotated relative to tabs 738 to secure the cap insert 730 in the cap 721. Additionally or alternatively, the cap insert 730 may be coupled to the cap 721 via one or more other suitable connection techniques.

In some cases, in order to equalize the pressure in the second container as water is flowing from the first container through the lumen 715 into the second container, an air vent 729 may be included in the cap insert 730. The air vent 729 may allow air to escape the second container, thereby preventing pressure build-up within the second container.

The cap insert 730 may include a biasing system 750 configured to actuate a sliding valve mechanism 733 which may include a first sliding valve 731 and a second sliding valve 732. The sliding valve mechanism 733 may be configured to move from a first valve position 740 (FIG. 10A) to a second valve position 745 (FIG. 10B) in response to a pressure at the sliding valve mechanism 733. The first sliding valve 731 and the second sliding valve 733 may be biased towards a first valve position 740 in which the first sliding valve 731 is biased towards the second sliding valve 732 via a first spring 734, and the second sliding valve 732 is biased towards the first sliding valve 731 via a second spring 735.

The first sliding valve 731 and the second sliding valve 732 may have any suitable shape. Example suitable shapes of the first sliding valve 731 and/or the second sliding valve 732 may include, but are not limited to, a rounded cross-sectional shape, a rectangular cross-sectional shape, an oval cross-sectional shape, a circular cross-sectional, a square cross-sectional shape, and/or other suitable shape. In one example, the first sliding valve 731 and/or the second sliding valve 732 may have a non-circular cross-sectional shape to ensure holes or openings therethrough remain aligned with the lumens or openings 715, 725, 726, 727, 729, 736 of or in communication with the insert 730. In some cases, an O-ring may be positioned around the cap insert 730 to prevent air from escaping the second container around the cap insert 730.

In some cases, the second container may be configured to receive a fluid (e.g., water) and/or other suitable fluid from the first container via the lumen 715 extending from the first portion 710 to the second portion 720 of the coupling mechanism 700 based on a pressure at the second container (e.g., second container 530) and/or other suitable container. For example, when an interior volume of the second container is depressurized, such as, in-between procedures, the sliding valve mechanism 733 may be in the first position 740. When the sliding valve mechanism 733 is in the first position 740, an opening 736 within the first sliding valve 731 may align with the lumen 715 of the coupling mechanism 700, thereby allowing a flow of fluid from a first container through the lumen 715 and into a second container. The cap insert 730 may define a volume of a space 760 within the second container. In some cases, the gas supply tubing 726 may allow a gas (e.g., air, CO₂) to flow from a gas supply (not explicitly shown) through the gas supply tubing 726 and into the interior volume of the second container. The gas may pressurize the space 760 at the sliding valve mechanism 733 where the first sliding valve 731 and the second sliding valve 732 meet, permitting the sliding valve mechanism 733 to move from the first valve position 740 (e.g., as shown in FIG. 10A) to the second valve position 745 (e.g., as shown in FIG. 10B). For example, when the pressure increases within the space 760, a bias force of the first spring 734 and the second spring 735 may be overcome, and the first sliding valve 731 and the second sliding valve 732 begin to move away from one another, the first spring 734 and the second spring 735 compress, and the sliding valve mechanism 733 moves to the second valve position 745, as shown in FIG. 10B. When the sliding valve mechanism 733 is in the second valve position 745, the lumen 715 is closed off from the second container coupled to the second portion 720 of the coupling mechanism 700, thereby stopping a flow of fluid through the lumen 715 of the coupling mechanism 700 from the first container to the second container.

An opening 737 in the second sliding valve 732 may align with the water supply tubing 727 when the sliding valve mechanism 733 is in the second valve position 745. This allows the gas to pressurize the fluid within the second container, forcing the fluid out through the water supply tubing 727 and through a lens wash supply tube/water supply tube.

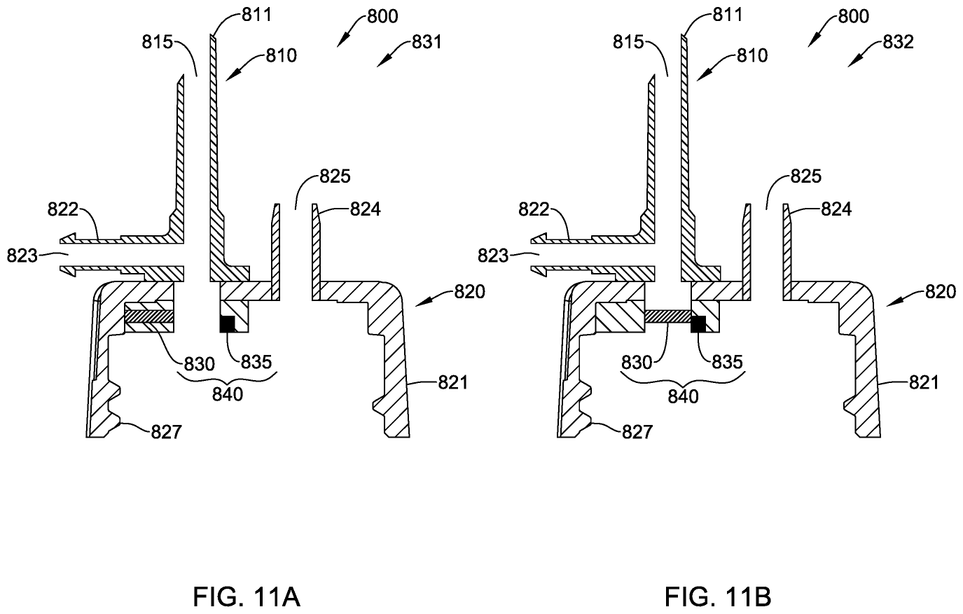

FIG. 11A depicts a cross-sectional view of an illustrative coupling mechanism 800 including a sliding mechanism 830 and FIG. 11B depicts a cross-sectional view of the illustrative coupling mechanism 800 including the sliding mechanism 830. The coupling mechanism 800 may include a first portion 810, a second portion 820, and a lumen 815 extending through the first portion 810 and the second portion 820. The coupling mechanism 800 may be configured to fluidly couple a first container (e.g., an IV bag and/or other suitable container) and a second container (e.g., a water bottle and/or other suitable container) via the lumen 815. In some cases, the second container may be configured to receive a fluid (e.g., water and/or other suitable fluid) from the first container via the lumen 815 extending from the first portion 810 to the second portion 820 of the coupling mechanism 800 based on a pressure at the second container (e.g., second container 530 and/or other suitable container).

An end 811 of the first portion 810 may include and/or may be a bag spike configured to engage with a spike port of an IV bag (e.g., first container 520 and/or other suitable container). The second portion 820 of the coupling mechanism 800 may be and/or may include a cap 821 configured to cover an opening of a container (e.g., second container 530 and/or other suitable container).

The cap 821 may be configured to couple to a container in any suitable manner. In some cases, the cap 821 may be configured to be coupled to a container via a snap fit, interference fit, a threaded connection, a heat seal to seal the container in a fluid and pressure tight manner, and/or other suitable connection techniques. In one example, the cap 821 may be configured to couple to a container, such as a bottle, via a thread fastening mechanism 827.

The cap 821 may be formed from any suitable materials including, but not limited to, polymers, metals, combinations of materials, and/or other suitable materials. In one example, the coupling mechanism 800 may be entirely or at least partially formed from include polycarbonate materials. Where polymers are selected as coupling mechanism 800 materials, the materials may have durometer in a range of about 75A to 90A Shore hardness, among other possible values.

The first portion 810 of the coupling mechanism 800 may include a first fluid port 822. The first fluid port 822 may include a lumen 823 that may be in fluid communication with the lumen 815. The first fluid port 822 may be configured to engage with a first tubing (e.g., irrigation supply tubing 525) and/or other suitable tubing. The second portion 820 of the coupling mechanism 800 may include a second fluid port 824. The second fluid port 824 may be in fluid communication with a second container via a lumen 825. The second fluid port 824 may be configured to engage with a second tubing (e.g., second tubing 515) and/or suitable tubing which may house a gas supply tubing (e.g., gas supply tubing 534) and a water supply tubing (e.g., water supply tubing 533), as discussed with reference to FIG. 5.

An actuating mechanism 840 (e.g., a biasing mechanism or other suitable actuating mechanism) may be configured to actuate a sliding valve 830 within the second portion 820 of the coupling mechanism 800 from a first valve position 831 (e.g., an opened valve position, as depicted in FIG. 11A) to a second valve position 832 (e.g., a closed valve position, as depicted in FIG. 11B). The actuating mechanism 840 may include a pressure sensor 835, the sliding valve 830, and/or one or more other suitable components. In some cases, when an interior volume of the second container is depressurized, the pressure sensor 835 senses the reduced pressure and the sliding valve 830 may be automatically moved to the first valve position 831, thereby allowing a flow of fluid through the lumen 815 of the coupling mechanism 800 from the first container to the second container. When the pressure sensor 835 senses that the fluid in the second container is pressurized, the sliding valve 830 may be automatically moved to the second valve position 832 in which the sliding valve 830 closes the lumen 815 relative to the second container, thereby stopping a flow of fluid through the lumen 815 of the coupling mechanism 800 from the first container to the second container. In other cases, when a user calls for irrigation at an endoscope, a suction through the first tubing pulls the fluid from the first container through the first fluid port 822, and the pressure sensor 835 may sense the change in pressure and closes the valve 830, thereby stopping a flow of fluid through the lumen 815 of the coupling mechanism 800 from the first container and into the second container, and redirecting the fluid through the first tubing to the endoscope.

In some cases, the gas supply tubing may allow a gas (e.g., air, CO₂) to flow from a gas supply (not explicitly shown) through the second port 824 and into the interior volume of the second container. This allows the gas to pressurize the second container, thereby closing the valve 830, and forcing the fluid out through the water supply tubing and through a lens wash supply/water supply tube.

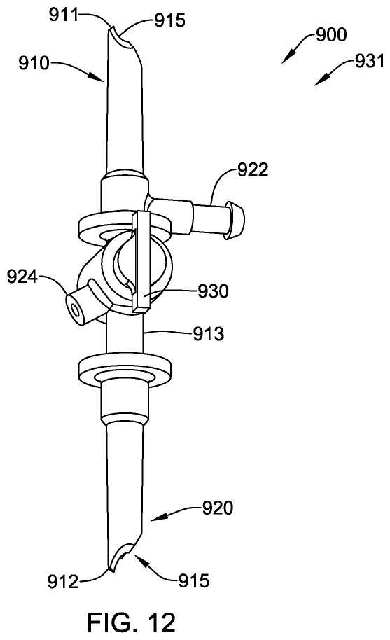

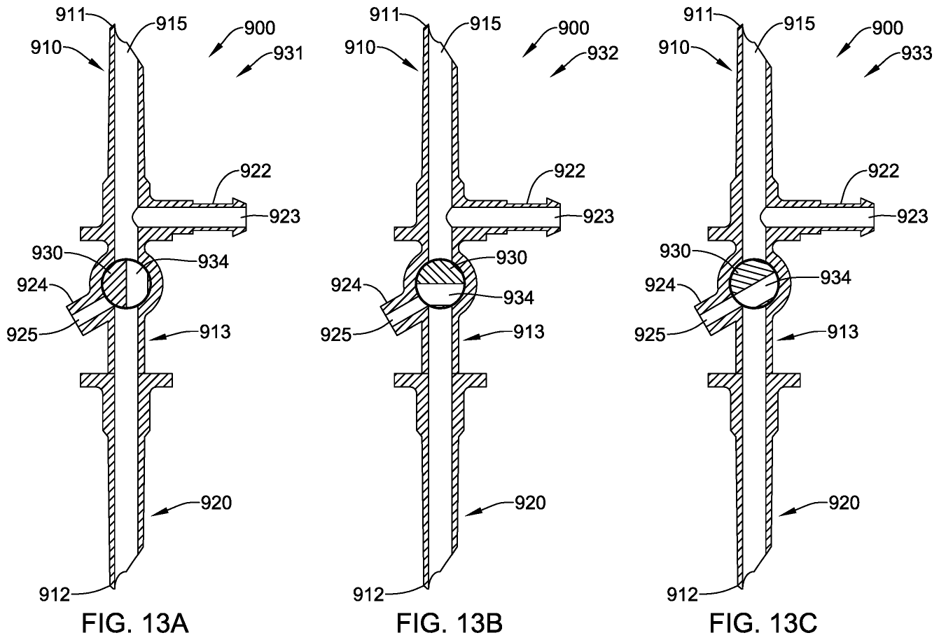

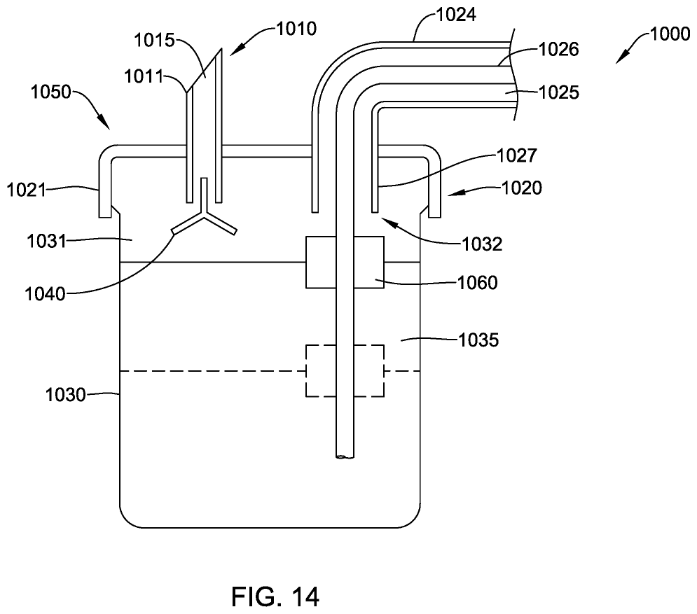

FIG. 12 depicts a schematic view of an illustrative coupling mechanism 900, FIG. 13A depicts a schematic cross-sectional view of the illustrative coupling mechanism 900, FIG. 13B depicts a schematic cross-sectional view of the illustrative coupling mechanism 900, and FIG. 13C depicts a schematic cross-sectional view of the illustrative coupling mechanism 900. The coupling mechanism 900 may include a first portion 910, a second portion 920, and a lumen 915 extending through the first portion 910 and the second portion 920.

The coupling mechanism 900 may be configured to fluidly couple the first container (e.g., an IV bag and/or other suitable container) and the second container (e.g., a second IV bag and/or other suitable container) via the lumen 915. In one example, a first end 911 of the first portion 910 may include and/or may be a bag spike configured to engage with a first container, (e.g., a spike port of an IV bag and/or other suitable container) and a second end 912 of the second portion 920 of the coupling mechanism 900 may be a second bag spike configured to engage with a second container, (e.g., a second spike port of a second IV bag and/or other suitable container). The second container may also include or may be coupled to a port for providing lens was supply and/or a water supply to an endoscope.

A middle portion 913 of the coupling mechanism 900 may include a first fluid port 922 and a second fluid port 924. The first fluid port 922 may include a lumen 923 that may be in fluid communication with the lumen 915. In some cases, the first fluid port 922 may be configured to engage with an irrigation supply tubing (e.g., irrigation supply tubing 525) and/or other suitable tubing. In some cases, the first fluid port 922 may be configured to engage with a third container (e.g., a spike port of an IV bag and/or other suitable container). In some cases, the second fluid port 924 may include a lumen 925 that may be in fluid communication with the lumen 915 of the coupling mechanism 900. In some cases, the second fluid port 924 may be part of a depressurizing valve and/or may be configured to couple to a depressurizing valve (not explicitly shown) via a luer lock, a barb fit, or any other suitable connection. In some cases, the second fluid port 924 may be configured to be coupled to a gas supply tubing to pressurize an interior volume of the second container via the lumen 915.

The middle portion 913 may further include an adjustable valve 930 configured to move between at least a first position associated with a first configuration 931 of the adjustable valve 930 (as shown, for example, in FIG. 13A), a second position associated with a second configuration 932 of the adjustable valve 930 (as shown, for example, in FIG. 13B), and a third position associated with a third configuration 933 of the adjustable valve 930 (as shown, for example, in FIG. 13C). The adjustable valve 930 may include a lumen 934 configured to be fluid communication with one or more ports of the coupling mechanism 900 as the adjustable valve 930 is adjusted.

When the adjustable valve 930 is in the first position at the first valve configuration 931, the lumen 934 within the adjustable valve 930 may align with the lumen 915, and a fluid may flow freely within the lumen 915 from the first portion 910 to the second portion 920 due to gravity and/or other suitable forces. When the adjustable valve 930 is in the second position at the second valve configuration 932, the adjustable valve 930 may be rotated such that the lumen 934 is no longer aligned with the lumen 915 and the adjustable valve 930 is closed such that the flow of fluid is blocked within the lumen 915 from traveling between the first portion 910, the second portion 920, and the second fluid port 924. When the adjustable valve 930 is in the third position at the third valve configuration 933, the adjustable valve 930 is rotated such that the lumen 934 is aligned with the lumen 925. When the lumen 934 is aligned with the lumen 925, the lumen 925 is in fluid communication with the lumen 915 and the second portion 920 of the coupling mechanism 900.

In some cases, when the second fluid port 924 is coupled to a gas supply line, the adjustable valve 930 may move to the third position at the third valve configuration 933 and a gas (e.g., air, CO₂) may flow through the lumen 925 into lumen 915, and through the second portion 920 of the coupling mechanism to a second container to pressurize the second container. This is just an example. In another example, when the adjustable valve 930 is in the third position at the third valve configuration 933, the lumen 925 may be used as a depressurizing lumen to expel gas from the second container coupled to the second portion 920 of the coupling mechanism 900.

The coupling mechanism 900 may be formed from any suitable material including, but not limited to, polymers, metals, combinations of materials, and/or other suitable materials. In one example, the coupling mechanism 900 may be entirely or at least partially formed from polycarbonate materials. Where polymers are selected as coupling mechanism 900 materials, the materials may have durometer in a range of about 75A to 90A Shore hardness, among other possible values.

In some cases, it may be desirable to refill the second container during a procedure or between procedures, as necessary. The coupling mechanism 900 may couple the second container to a first container filled with a fluid, thereby increasing the amount of fluid available. In some cases, when the first container is an IV bag, the first container can be replaced as often as needed, without replacing the second container/water bottle. This eliminates the need to disconnect the second container from tubing throughout the day eliminating or greatly reducing the possibility of cross contamination by removing the need to replace the water container.

FIG. 14 depicts a schematic view of an illustrative fluid supply system 1000 for an endoscope (e.g., endoscope 510). The fluid supply system 1000 may include a first container (not explicitly shown) having an interior volume configured to contain a fluid (e.g., water). The fluid supply system 1000 may include a second container 1030 having an interior volume 1031 which may be configured to contain a fluid 1035 (e.g., water).

The first container may be fluidly coupled to the second container 1030 via a coupling mechanism 1050. In some cases, the coupling mechanism 1050 may include a first portion 1010, a second portion 1020, and a lumen 1015 extending between the first portion 1010 and the second portion 1020. An end 1011 of the first portion 1010 of the coupling mechanism 1050 may be configured to engage with a spike port positioned at the first container. For example, the first portion 1010 may include a bag spike which may be configured to be inserted into the spike port of the first container. The second portion 1020 of the coupling mechanism 1050 may include a cap 1021 that may be configured to cover an opening of the second container 1030.

The cap 1021 at the second portion 1020 may be fitted for the second container 1030 (e.g., bottle and/or other suitable container). The cap 1021 may be configured to couple to the second container 1030 in any suitable manner. In one example, the cap 1021 may be configured to couple to the second container 1030 via a threaded fastening mechanism and/or other suitable coupling mechanism.

The second portion 1020 of the coupling mechanism 1050 may include a fluid port 1024. The fluid port 1024 may be in fluid communication with the interior volume 1031 of the second container 1030. Similar to as discussed elsewhere herein, a first end of a second tubing may be coupled to the fluid port 1024, and a second end of the second tubing may be coupled to the endoscope. The second tubing may be considered to be a shared tubing, which may include a dual lumen. The second container 1030 may be connected in fluid communication with the endoscope via a shared length of gas supply tubing 1025 and a lens wash supply/water supply tubing 1026. The shared gas supply tubing 1025 extends from the fluid port 1024 in the second portion 1020 of the coupling mechanism 1050.

The first container may be in fluid communication with the second container 1030 via the lumen 1015 of the coupling mechanism 1050. In some cases, the second container 1030 may be configured to receive a fluid (e.g., water) from the first container via the lumen 1015 extending from the first portion 1010 to the second portion 1020 of the coupling mechanism 1050 based on a pressure at the second container 1030. For example, when the interior volume 1031 of the second container 1030 is depressurized, such as, in-between procedures, a valve 1040 within the second portion 1020 of the coupling mechanism 1050 gives way to the fluid flowing through the lumen 1015 of the coupling mechanism 1050 from the first container and into the second container 1030. In some cases, the valve 1040 may be an umbrella valve, a duckbill valve, a one-way valve, a dome valve, or any other suitable valve.

When the fluid in the second container 1030 is pressurized, the pressure within the interior volume 1031 of the second container 1030 increases and the valve 1040 closes, thereby stopping a flow of fluid through the lumen 1015 of the coupling mechanism 1050 from the first container and into the second container 1030. When a user calls for irrigation at the endoscope, a suction through the water supply tubing 1026 pulls the fluid from the second container 1030 and through the fluid port 1024, and the suction closes the valve 1040, thereby stopping a flow of fluid through the lumen 1015 of the coupling mechanism 1050 from the first container and into the second container 1030.

In some cases, the gas supply tubing 1025 may allow a gas (e.g., air, CO₂) to flow from a gas supply (not explicitly shown) through the gas supply tubing 1025 and into the interior volume 1031 of the second container 1030. This allows the gas to pressurize the fluid 1035 within the second container 1030, thereby closing the valve 1040, and forcing the fluid out through the water supply tubing 1026 and through a lens wash tube and/or an irrigation supply tube.

In some cases, a floating stop valve 1060 may be positioned within the interior volume 1031 of the second container 1030, adjacent to the water supply tubing 1026 to prevent overfilling of the second container 1030 and fluid from entering a lumen of the gas supply tubing 1025. For example, the floating stop valve 1060 may include a size and shape to fit across an end 1027 of the fluid port 1024. When the fluid within the second container 1030 rises due to the volume of the second container 1030 being filed from fluid from the first container via the lumen 1015, the floating stop valve 1060 will engage the end 1027 of the fluid port 1024 and block fluid from flowing into the gas supply tubing 1025. In some cases, when the fluid within the second container 1030 is lower, the floating stop valve 1060 will descend with the fluid level, as indicated by the dashed lines in FIG. 14.

As will be appreciated, the lengths of irrigation, lens wash, gas supply, alternate gas supply tubing may have any suitable size (e.g., diameter). In addition, the sizing (e.g., diameters) of the tubing may vary depending on the application.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A fluid supply system for an endoscope, the fluid supply system comprising:
a first container having an interior volume configured to contain a fluid;
a second container having an interior volume configured to contain a fluid; and
a coupling mechanism having a first portion and a second portion, and a lumen extending therebetween, wherein the first portion of the coupling mechanism is configured to engage with the first container and the second portion is configured to engage with the second container; and
wherein the first portion includes a first fluid port in fluid communication with the first container via the lumen; and
wherein the first container and the second container are in fluid communication via the lumen.

2. The fluid supply system of claim 1, wherein the first container is an intravenous (IV) bag, and an end of the first portion of the coupling mechanism is a bag spike configured to be coupled to the IV bag.

3. The fluid supply system of claim 1, wherein the coupling mechanism is a cap configured to cover an opening of the second container.

4. The fluid supply system of claim 3, wherein the first fluid port is configured to engage with a first end of a first tubing, the first tubing having a second end configured to engage with the endoscope.

5. The fluid supply system of claim 4, wherein the second portion of the coupling mechanism includes a second fluid port configured to engage with a first end of a second tubing, the second tubing having a second end configured to engage with a fluid supply.

6. The fluid supply system of claim 3, wherein the cap includes a first spring and a second spring configured to actuate a sliding valve mechanism, the sliding valve mechanism configured to move from a first valve position to a second valve position.

7. The fluid supply system of claim 1, wherein the second container is configured to receive a fluid from the first container through the lumen extending from the first portion to the second portion of the coupling mechanism based on a pressure at the second container.

8. The fluid supply system of claim 1, wherein the first container is a first intravenous (IV) bag, and an end of the first portion of the coupling mechanism includes a first bag spike configured to be coupled to the first IV bag, and wherein the second container is a second IV bag and an end of the second portion of the coupling mechanism includes a second bag spike configured to be coupled to the second IV bag.

9. The fluid supply system of claim 8, wherein a middle portion of the coupling mechanism includes the first fluid port, the middle portion positioned between the first portion and the second portion, wherein the first fluid port is in fluid communication with the first container, the second container, and a third container via the lumen, and wherein the third container is configured to contain a fluid.

10. The fluid supply system of claim 8, wherein the coupling mechanism further includes an adjustable valve positioned between the first portion and the second portion of the coupling mechanism, the adjustable valve is configured to be adjusted to control a flow of fluid through the lumen extending between the first portion and the second portion.

11. A coupling mechanism for an endoscope comprising:
a first portion configured to engage with a first container;
a second portion configured to engage with a second container;
a lumen extending from the first portion to the second portion;
a first fluid port of the first portion, the first fluid port in fluid communication with the lumen; and
a second fluid port of the second portion, the second fluid port is configured to be in fluid communication with a volume defined by the second container when the second portion engages the second container.

12. The coupling mechanism of claim 11, wherein the second portion includes a valve configured to control a flow of fluid through the lumen extending from the first portion to the second portion.

13. The coupling mechanism of claim 11, wherein the first container is an intravenous (IV) bag, and the first portion includes a bag spike configured to be coupled to the IV bag.

14. The coupling mechanism of claim 11, further comprising:
a cap and the cap includes the first portion and the second portion.

15. The coupling mechanism of claim 14, wherein the cap includes a biasing system configured to actuate a sliding valve mechanism, the sliding valve mechanism is configured to move from a first position to a second position in response to a pressure at the sliding valve mechanism, wherein the biasing system biases the sliding valve mechanism to the first position.

16. The coupling mechanism of claim 15, wherein a fluid from the second fluid port pressurizes a space at the sliding valve mechanism, thereby causing the sliding valve mechanism to move from the first position to the second position.

17. A fluid supply system for an endoscope, the fluid supply system comprising:
a first container having an interior volume configured to contain a fluid;
a second container having an interior volume configured to contain a fluid; and
a coupling mechanism having a first portion and a second portion, and a lumen extending therebetween, wherein the first portion of the coupling mechanism is configured to engage with the first container and the second portion is configured to engage with the second container;
wherein the first container and the second container are in fluid communication via the lumen;
wherein the first portion of the coupling mechanism further includes a first fluid port configured to engage with a first end of a first tubing, the first tubing having a second end configured to engage with the endoscope; and
wherein the second portion of the coupling mechanism further includes a second fluid port configured to engage with a first end of a second tubing, the second tubing having a second end configured to engage with a fluid supply.

18. The fluid supply system of claim 17, wherein the second portion includes a valve configured to control a flow of fluid through the lumen.

19. The fluid supply system of claim 18, wherein the valve includes a sliding mechanism and the sliding mechanism is configured to be actuated between a first position and a second position in response to a sensed pressure at the second portion of the coupling mechanism.

20. The fluid supply system of claim 18, wherein:
the valve includes a biasing system configured to actuate a sliding mechanism, the sliding mechanism is configured to move from a first position to a second position in response to a pressure at the sliding mechanism,
the biasing system biases the sliding mechanism to the first position,
the first position is configured to allow fluid to flow between the first container and the second container via the lumen, and
the second position is configured to block fluid flow between the first container and the second container via the lumen and allow fluid to flow from the second container through the second portion.
